(19) United States
(12) Patent Application Publication
(10) Pub. No.: US 2024/0029850 A1
(43) Pub. Date: Jan. 25, 2024

(54) METHOD AND SYSTEM UTILIZING MACHINE LEARNING TO DEVELOP AND IMPROVE CARE MODELS FOR PATIENTS IN AN ELECTRONIC PATIENT SYSTEM

(71) Applicant: Opeeka, Inc., Folsom, CA (US)

(72) Inventors: Kate Duggento CORDELL, El Dorado Hills, CA (US); Jacob William JORGENSEN, FOLSOM, CA (US)

(21) Appl. No.: 18/356,992

(22) Filed: Jul. 21, 2023

Related U.S. Application Data
(60) Provisional application No. 63/391,680, filed on Jul. 22, 2022.

(51) Int. Cl. G16H 20/00; G16H 50/70; G16H 10/60

(52) U.S. Cl. CPC G16H 20/00; G16H 50/70; G16H 10/60

(57) ABSTRACT

Server-implementing methods include receiving a selection of records for patients from an external source coupled to the server, and vectorizing at least set of healthcare record items associated with the selected patients and other patients from the data source. At least one vector element may be determined from healthcare record items for each patient. Vectors may be formed from at least one of the healthcare record items and the separate vectors concatenated together to form final vectors for the patients. A similarity search may be performed using the final vectors to determine a group of similar patients from the vectorized patients of the system. Selected patients that are within a same dimensional space as a focal patient may be labelled in the batch and presented on computer display. Further, intake of patient data from non-medical record sources may be automated and facilitated through the use of a large language model.

### RELATED APPLICATIONS

This application claims the benefit of priority to U.S. Provisional Patent Application No. 63/391,680 entitled “Method And System Utilizing Machine Learning To Develop And Improve Care Models For Patients In An Electronic Patient System” filed Jul. 22, 2022, the entire contents of which are incorporated herein by reference for all purposes.

### BACKGROUND

Present electronic patient systems require system administrators to manually label patients in digital databases. These administrators typically use the labeled patient databases to assist clinicians to compare the treatment histories of particular patients of interest via a search query, or by making selections from provided lists (e.g., patient type, primary complaint, patient care history, etc.) The terms of the search query or the selections from the list are used to find patients that have labels that match the terms of the query or the selections.

### SUMMARY

Various aspects include systems and methods to create a machine learning model for patient ontology. Various aspects may also provide systems and methods for administrators to label data for the machine learning model, and for the model to learn substitute patient categories.

Various aspects may train the machine learning model to classify patients and/or label patients for an electronic database. Clinicians may use the machine learning model to form groups of similar patients, label a plurality of the similar patients at once, and/or dynamically add new classes of patients into the system. Various aspects include methods for dynamically retraining the machine learning model as data is being labeled. Thus, patient labeling using the machine learning model may become easier as the model improves, and the model may be used to label groups of patients quickly.

In some embodiments, a large language machine language model may also be used in conjunction with the machine learning model to receive and recognize pertinent information in patient records, and narrative input from patients, patient caretakers, and others on behalf of patients, particularly non-standard and non-medical records, and format such information for application to and processing by the machine learning model of various embodiments.

### BRIEF DESCRIPTION OF THE DRAWINGS

The accompanying drawings, which are incorporated herein and constitute part of this specification, illustrate exemplary embodiments of the claims, and together with the general description given and the detailed description, serve to explain the features herein.

FIG. 1 is a system block diagram illustrating an example system suitable for implementing various embodiments.

FIG. 2 is a process flow diagram of an example method 200 for labeling patients in an electronic patient system in accordance with various embodiments.

FIG. 3 is a process flow diagram of another example method 300 based on the method 200 in accordance with some embodiments.

FIG. 4 is a process flow diagram of another example method 400 based on the method 200 in accordance with various embodiments.

FIG. 5 is a process flow diagram of another example method 500 based on the method 200 in accordance with various embodiments.

FIG. 6 is a component block diagram of a server suitable for use with various embodiments.

### DETAILED DESCRIPTION

Various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the claims.

Various embodiments include methods and a server or other computing system implementing the methods for labeling patients in an electronic patient system. Embodiment methods are described that use machine learning techniques to compare patient representations for similar patients, thereby allowing clinicians to make better choices for care planning and treatment alternatives using knowledge of the history and experience of similar patients who may have similar histories, conditions, or diagnoses.

Various embodiment methods are configured to use trained machine learning models to link and merge data from multiple sources that contain health information (including physical health, behavioral health, mental health, and social health) in a manner that provides a holistic view of patient circumstances and history. Embodiment methods are configured to enable the controlled sharing of patient information between clinicians that act as part of a care team and the patient through web and mobile applications. Embodiment methods are configured to unify information into a comprehensive story about the patient enabling all clinicians to collaborate in a virtual setting for planning patient care. Embodiment methods are configured to automate the workflow of data ingestion from health care records and mobile devices with controlled sharing to ease the burden of measurement-based care.

In addition to using trained machine learning models to link and merge patient health information, a large language model or neural network may be trained and/or fine-tuned to automate the receipt and processing of information about patients from a range of sources beyond standard diagnostic and patient intake forms, including reformatting, organizing or summarizing such information for receipt and use by the trained machine learning models of various embodiments. Using the context and language understanding capabilities of large language neural network models may enable various embodiments to take in, organize and apply information from a wide variety of sources beyond standard medical forms in a manner that may benefit clinicians in patient care planning and diagnostics.

Various embodiments provide benefits to the treatment of patients by providing an automated system for performing patient population analytics to inform health system functioning. By categorizing patients across many aspects of health status, lifestyle, life experiences, and social interactions, various embodiments may enable clinicians to identify other patients with very similar backgrounds and conditions so that treatment plans and the results of treatments on such similar patients can be considered in treatment planning and care management.

Various embodiments include a server/computer implemented methods for receiving a selection of records for patients from an external source coupled to the server/computer, and vectorizing at least a set of healthcare record items associated with the selected patients and other patients from the data source. At least one of the various vector elements may be determined by the server from healthcare record items for each patient. In some embodiments, records of patients may be received from non-health care sources (e.g., social services, Social Security, foster care, social care, school, residential services, home care, home health, and hospice services, police, military, court and other record sources) may also be received, such as processed by a trained or fine-tuned large language neural network model, with information that could be useful in classifying populations of patients included in vectorized record items. The server may form vectors from at least one of the healthcare record items, as well as other record items, and concatenate the separate vectors together to form final vectors for the patients. A similarity search may be performed by a trained machine learning module executing on the server using the final vectors to determine a group of similar patients from the vectorized patients of the system. Selected patients that are within a same dimensional space as a focal patient (i.e., a particular patient, such as a patient under treatment) may be labelled in a batch or batches. Once labeled, patient data may be presented on computer displays in formats and organizations that may help clinicians, care workers, and social workers to better treat patients.

Various embodiments may be used for patient recommendations. In particular, the machine learning systems and methods may be used recommending patients that are similar to a selected patient, and/or for digital twin patient recommendations. This may enable recommending or assessing one or more treatment plans for a selected patient based on comparisons of treatment plans and results observed for similar, complementary or digital twin patients.

In order for typical machine learning models to perform with high accuracy, an administrator may label a portion of the patient database, which can be time consuming. Various embodiments provide a data labeling system for patient ontology which uses machine learning to quicken the process for labeling data, such as labeling patients in an electronic database. A user and/or administrator may use the machine learning technologies of various embodiments to label patient data quickly and intuitively to improve the patient recommendations and/or digital twin recommendations provided. Also as the administrator labels patient data, the machine learning model may be dynamically retrained. By retraining the model, the performance of the data labeling system may be improved over time, as the system uses the machine learning model to label large batches of similar patients.

Various embodiments may allow for the machine learning model to learn labeling of a patient database in less time than current systems. This may provide for an increased number of patients to be labeled within a predetermined period of time. That is, various embodiments may reduce the time, complexity, and computing resources for data on-boarding and/or labeling processes. The increased number of patients accurately labeled may improve the performance for patient and/or digital twin recommendations.

As noted, in various embodiments, the machine learning model may be trained to vectorize patient data within medical databases, and identify similar and digital twin patients within a patient database for comparison to and managing treatment plans for a selected patient. However, information contained in other sources of incident, behavior and life-experience records on individuals may also be useful in identifying similar and digital twin patients for an individual, as life experiences can impact how individuals respond to various treatment plans, particularly for behavioral conditions and medical conditions resulting from environmental exposures. Sources of such information and records of individuals varies widely, and may include school records, military service records, employment records, social services records, law enforcement records, court records, financial and credit score records, and many more. A challenge faced by clinicians attempting to understand and treat the whole person is that such other non-medical records are not kept in formats compatible with medical evaluation systems. This requires clinicians to play detective and spend time reviewing records that are not available in modern medical practice.

To address this issue, some embodiments further employ a large language model (sometimes referred to as “artificial intelligence” or “AI” models) that is trained or fine-tuned to receive as an input (e.g., access via electronic databases) personal records of individuals from a variety of sources in any format, recognize information about the person in the records that could be relevant to medical treatments and particularly useful for matching to similar or digital twin patients, extract such information, and present the relevant information in a format that is consistent with or suitable for processing by the machine learning models of various embodiments. This capability thus may automate the intake, organizing and processing of clinically relevant information about patients received from non-medical databases for inclusion within patient categorizing and matching systems. The information can then be used to complete various virtual assessment forms that may be created to store and process a profile of the patient at a particular point in time and can be compared with other assessments done at other times and serve as a basis for determining future appropriate treatment plans for the patient. These virtual assessments can be created by or on behalf of a healthcare organization, a payer organization, or third parties skilled and experienced in developing and promoting such assessments.

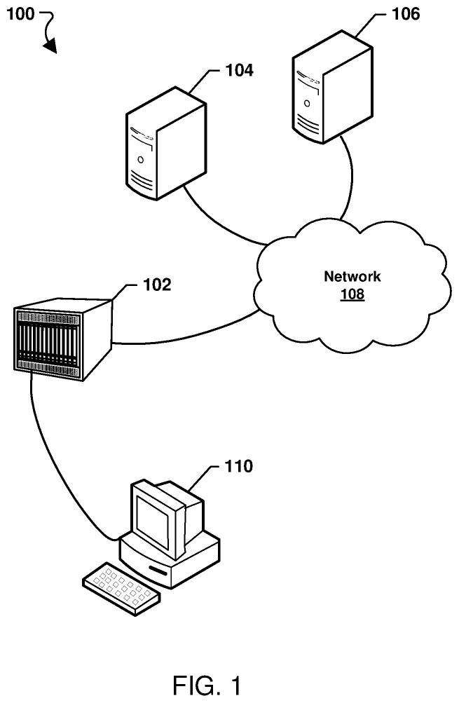

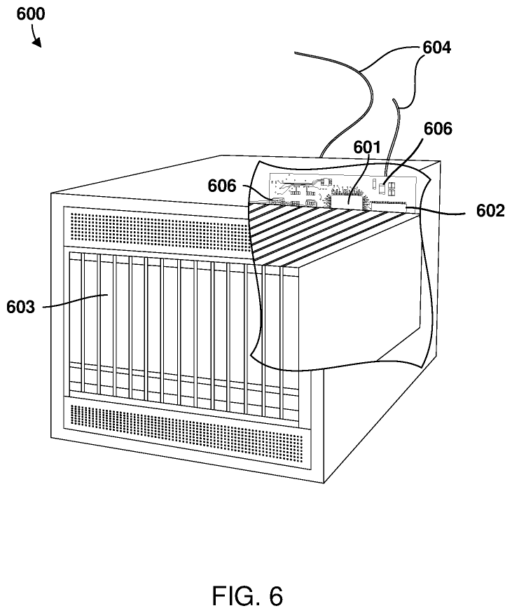

FIG. 1 is a system block diagram illustrating an example electronic patient system 100 suitable for implementing the various embodiments. The system 100 may include a server 102 that may communicate with a number of patient record databases 104, 106 via a network (e.g., the Internet, a hospital network, a local area wired and wireless network, etc.), and one or more computers 110 having a display. As illustrated in more detail in FIG. 6, the server may include a processor coupled to memory and network interface circuitry for communicating data to and from patient record databases 104, 106 and the one or more computers 110 via the network 108. The server processor may be configured with processor-executable instructions to perform operations of various embodiments, with the processor-executable instructions stored on the memory, which is a non-transitory processor-readable medium.

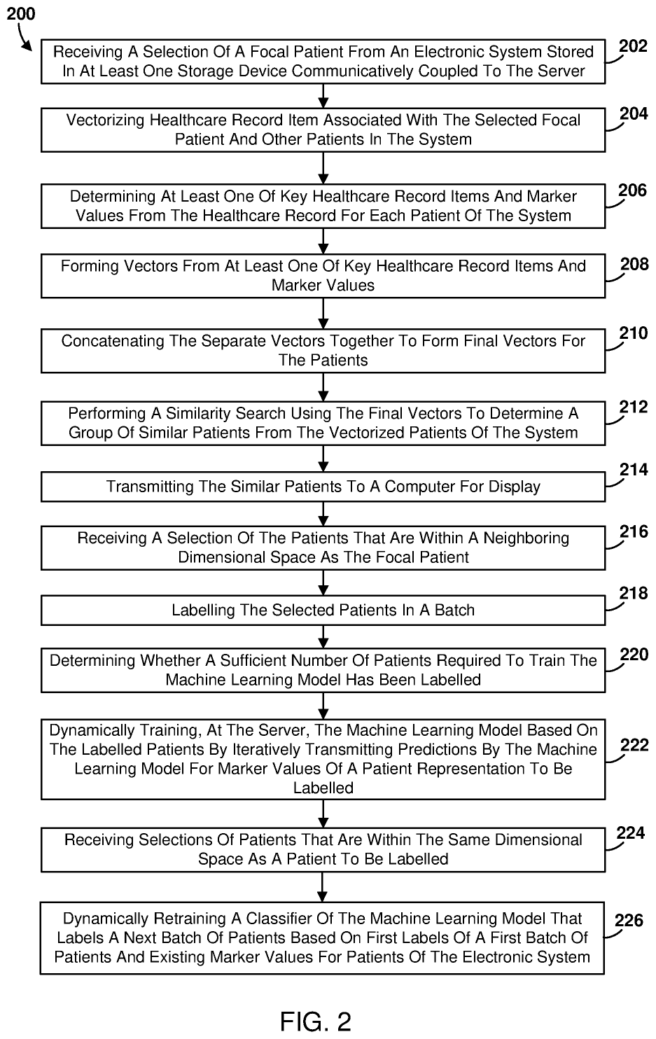

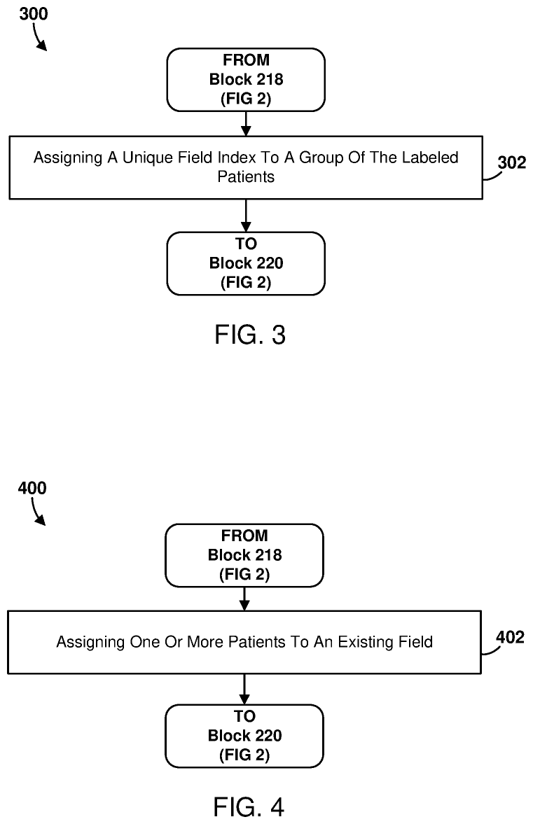

FIG. 2 is a process flow diagram of an example method 200 performed by a processor of server of an electronic patient system in accordance with various embodiments.

In block 202, the server may perform operations including receiving a selection of a focal patient from an electronic system stored in at least one storage device communicatively coupled to the server. In some embodiments, the server may receive a randomly selected patient from the electronic system.

In block 204, the server may perform operations including vectorizing at least one healthcare record item associated with the selected focal patient and other patients in the system. In some embodiments, the selected focal patient and the other patients in the system are without slot labels. In some embodiments, the dynamic number of slot labels may be used to label one or more patients, with different patients having different slot labels. In some embodiments, a machine learning model executing on the server initially may be untrained in labeling the one or more patients with the slot labels.

In some embodiments, vectorizing at least one healthcare record item associated with the selected focal patient and other patients in the system in block 204 may include vectorizing the at least one of the healthcare record items associated with the selected focal patient and other patients in the system using all the statistical characteristics taken together (minimum, maximum, variance, skewness, range, mean, mode, median) of the sequence of a patient's data elements to extract new insights that are not apparent from the individual longitudinal raw data points.

In some embodiments, vectorizing at least one healthcare record item associated with the selected focal patient and other patients in the system in block 204 may include vectorizing columns of the at least one of the healthcare record items associated with the selected focal patient and other patients in the system by at least one selected from a group including patient type, patient description, patient diagnosis, treatment type, treatment length, treatment description, and treatment category name list; and concatenating the vectorized columns together to form final vectors of the focal patient and other patients.

In block 206, the server may perform operations including determining at least one of key healthcare record items and marker values from the healthcare record for each patient of the system.

In block 208, the server may perform operations including forming vectors from at least one of key healthcare record items and marker values. This vectoring of healthcare record items and marker values transforms such information into a format that can be processed by the computing system.

In block 210, the server may perform operations including concatenating the separate vectors together to form final vectors for the patients.

In block 212, the server may perform operations including performing a similarity search using the final vectors to determine a group of similar patients from the vectorized patients of the system. For example, the computing system may use comparison algorithms that determine a difference, such as a vector distance between or dimensional space of the vector of a selected focal patient and the other vectors in the database, to identify those patient vectors with a minimal difference of separation from the vector of the selected focal patient.

In block 214, the server may perform operations including transmitting, via a communication network coupled to the server, the similar patients to a computer for display. This display may be configured to present the information on the similar patients to a clinician in a useful manner.

In block 216, the server may perform operations including receiving a selection of the patients that are within a neighboring dimensional space as the focal patient. In this manner, the server may receive selection inputs from a clinician identifying displayed similar patents or patient vectors that could be useful for training the system.

In block 218, the server may perform operations including labelling the selected patients in batch, wherein the selection of the patients and the focal patient within the same slots have similar slot labels.

In block 220, the server may perform operations including determining whether a sufficient number of patients required to train the machine learning model has been labelled. In some embodiments, the sufficient number of patients is at least partially based on at least one selected from a number of the patient record items, a measure of patient health heterogeneity, and/or a number of patients.

In block 222, the server may perform operations including, in accordance with a dynamic determination that the sufficient number of patients has been labelled, dynamically training the machine learning model based on the labelled patients by iteratively transmitting predictions by the machine learning model for marker values of a patient representation to be labelled.

In block 224, the server may perform operations including receiving selections of patients that are within the same dimensional space as a patient to be labelled.

In block 226, the server may perform operations including dynamically retraining a classifier of the machine learning model that labels a next batch of patients based on first labels of a first batch of patients and existing marker values for the patients of the electronic system.

FIG. 3 is a process flow diagram of an example method 300 performed by a processor of server of an electronic patient system in accordance with various embodiments. The method 300 includes the operations in blocks 202-218 of the method 200 as described above.

In block 302, the server may perform operations including assigning a unique field index to a group of the labeled patients.

After performing the operations in block 302, the server may perform the operations in blocks 220-226 of the method 200 as described above.

FIG. 4 is a process flow diagram of an example method 400 performed by a processor of server of an electronic patient system in accordance with various embodiments.

In block 402, the server may perform operations including assigning a unique field index to a group of the labeled patients.

After performing the operations in block 402, the server may perform the operations in blocks 220-226 of the method 200 as described above.

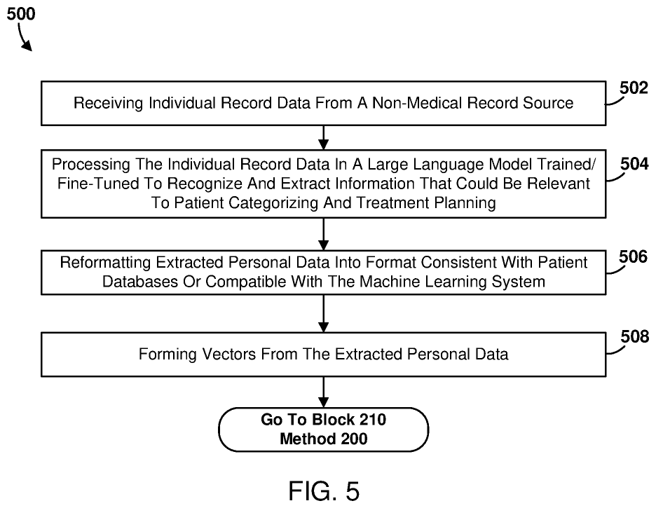

FIG. 5 is a process flow diagram of an example method 200 performed by a processor of server of an electronic patient system in accordance with various embodiments.

In block 502, the server may perform operations including receiving Individual record data from a non-medical record sources. For example, the server may receive files (e.g., loaded via transferrable media or via internetwork connections) from sources including school records, military service records, employment records, social services records, foster care, social care, residential services, school, home care, home health, hospice services, law enforcement records, court records, financial and credit score records, and the like.

In block 504, the server may perform operations including processing the individual record data in a large language model trained/fine-tuned to recognize and extract information that could be relevant to patient categorizing and treatment planning. The language processing capabilities of large language models may be fine-tuned to understand the meaning, context and importance of language in various forms of records written by individuals and under circumstances very different from and incompatible with medical records and patient data gathering. Such models may be fine-tuned to understand and thus extract (e.g., summarize or translate) information that has or could have clinical significance and/or be useful in identifying similar and digital twin patients. For example, school records could provide information on the level of education individuals attained while military records may reveal information regarding exposures to hazardous environments, noise levels and materials, as well as service-related injuries. Similarly, law enforcement and social service records may reveal information useful for diagnosing and treating behavioral matters.

In block 506, the server may perform operations including reformatting extracted personal data into format consistent with patient databases or compatible with the machine learning system. In addition to recognizing such information, the trained large language model may generate summaries or recast records that capture the medically-relevant information in a format conducive to processing by embodiment machine learning methods described herein.

In block 508, the server may perform operations including forming vectors from the extracted personal data. In such operations the machine learning models, with the assistance of the language processing capabilities of the trained large language model, may vectorize the data obtained from such non-medical record sources in a manner consistent with the operations performed in block 208 of the method 200 as described.

After performing the operations in block 508, the server may perform the operations in blocks 210-226 of the method 200 as described above.

FIG. 6 is a component block diagram of a server 600 suitable for use with various embodiments. With reference to FIGS. 1-6, various embodiments (including, but not limited to, embodiments described with reference to FIGS. 2-4) may be implemented on a variety of computing devices, including a server 600 as illustrated in FIG. 6. A server 600 may include a processor 601 coupled to volatile memory 602 and a large capacity nonvolatile memory, such as a disk drive 603. The processor 601 is configured with processor-executable instructions that are stored on a non-transitory processor-readable medium, such as a peripheral memory access device such as a floppy disc drive, compact disc (CD) or digital video disc (DVD) drive 606 coupled to the processor 601.

The server 600 may also include network access ports 604 (or interfaces) coupled to the processor 601 for establishing data connections with a network, such as the Internet and/or a local area network coupled to patient databases and servers, as well as computers with displays. The server 600 may include one or more transceivers 605 for communicating with wireless networks. The server 600 may include additional access ports, such as USB, Firewire, Thunderbolt, and the like for coupling to peripherals, external memory, or other devices.

Various embodiments illustrated and described are provided merely as examples to illustrate various features of the claims. However, features shown and described with respect to any given embodiment are not necessarily limited to the associated embodiment and may be used or combined with other embodiments that are shown and described. Further, the claims are not intended to be limited by any one example embodiment. For example, one or more of the operations of the methods may be substituted for or combined with one or more operations of the methods.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the claims.

In one or more embodiments, the functions described may be implemented in hardware, software, firmware, or any combination thereof. If implemented in software, the functions may be stored as one or more instructions or code on a non-transitory computer-readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the claims. Various modifications to these embodiments will be readily apparent to those skilled in the art, and the generic principles defined herein may be applied to other embodiments without departing from the scope of the claims. Thus, the present disclosure is not intended to be limited to the embodiments shown herein but is to be accorded the widest scope consistent with the following claims and the principles and novel features disclosed herein.

What is claimed is:

1. A method comprising:
receiving, at a server, a selection of a focal patient from an electronic system stored in at least one storage device communicatively coupled to the server;
vectorizing at least one healthcare record item associated with the selected focal patient and other patients in the system, wherein the selected focal patient and the other patients in the system are without slot labels, and wherein the a dynamic number of slot labels are used to label one or more patients, with different patients having different slot labels, and wherein a machine learning model at the server is untrained in labeling the one or more patients with the slot labels;
determining, at the server, at least one selected from the group including key healthcare record items and marker values from the healthcare record for each patient of the system;
forming, at the server, vectors from at least one selected from the group including key healthcare record items and marker values;
concatenating, at the server, the separate vectors together to form final vectors for the patients;
performing, at the server, a similarity search using the final vectors to determine a group of similar patients from the vectorized patients of the system;
transmitting, via a communication network coupled to the server, the similar patients to a computer for display;
receiving, at the server, a selection of the patients that are within a neighboring dimensional space as the focal patient;
labelling, at the server, the selected patients in batch, wherein the selection of the patients and the focal patient within the same slots have similar slot labels;
determining, at the server, whether a sufficient number of patients required to train the machine learning model has been labelled, wherein the sufficient number of patients is at least partially based on at least one selected from the group including a number of the patient record items, a measure of patient health heterogeneity, and a number of patients;
in accordance with a dynamic determination that the sufficient number of patients has been labelled, dynamically training, at the server, the machine learning model based on the labelled patients by iteratively transmitting predictions by the machine learning model for marker values of a patient representation to be labelled;
receiving selections of patients that are within the same dimensional space as a patient to be labelled; and
dynamically retraining, at the server, a classifier of the machine learning model that labels a next batch of patients based on first labels of a first batch of patients and existing marker values for the patients of the electronic system.

2. The method of claim 1, wherein receiving a selection of a focal patient from an electronic system comprises receiving, at the server, a randomly selected patient from the electronic system.

3. The method of claim 1, wherein vectorizing at least one healthcare record item associated with the selected focal patient and other patients in the system comprises vectorizing the at least one of the healthcare record items associated with the selected focal patient and other patients in the system using all the statistical characteristics taken together (minimum, maximum, variance, skewness, range, mean, mode, median) of the sequence of a patient's data elements to extract new insights that are not apparent from the individual longitudinal raw data points.

4. The method of claim 1, wherein vectorizing at least one healthcare record item associated with the selected focal patient and other patients in the system comprises vectorizing columns of the at least one of the healthcare record items associated with the selected focal patient and other patients in the system by at least one selected from a group including patient type, patient description, patient diagnosis, treatment type, treatment length, treatment description, and treatment category name list; and concatenating the vectorized columns together to form final vectors of the focal patient and other patients.

5. The method of claim 1, further comprising assigning a unique field index to a group of the labeled patients.

6. The method of claim 1, further comprising assigning one or more patients to an existing field.

7. The method of claim 1, further comprising:
receiving individual record data from a non-medical record source;
processing the individual record data in a large language model trained/fine-tuned to recognize and extract information that could be relevant to patient categorizing and treatment planning;
reformatting extracted personal data into format consistent with patient databases or compatible with the machine learning system; and
forming vectors from the extracted personal data.

8. A server, comprising:
a memory; and
a processor coupled to the memory and configured with processor-executable instructions to:
receive a selection of a focal patient from an electronic system stored in at least one storage device communicatively coupled to the server;
vectorize at least one healthcare record item associated with the selected focal patient and other patients in the system, wherein the selected focal patient and the other patients in the system are without slot labels, and wherein the a dynamic number of slot labels are used to label one or more patients, with different patients having different slot labels, and wherein a machine learning model at the server is untrained in labeling the one or more patients with the slot labels;
determine at least one selected from the group including key healthcare record items and marker values from the healthcare record for each patient of the system; from vectors from at least one selected from the group including key healthcare record items and marker values;
concatenate the separate vectors together to form final vectors for the patients;
perform a similarity search using the final vectors to determine a group of similar patients from the vectorized patients of the system;
transmit, via a communication network coupled to the server, the similar patients for display; receive a selection of the patients that are within a neighboring dimensional space as the focal patient;
label the selected patients in batch, wherein the selection of the patients and the focal patient within the same slots have similar slot labels;
determine whether a sufficient number of patients required to train the machine learning model has been labelled, wherein the sufficient number of patients is at least partially based on at least one selected from the group including a number of the patient record items, a measure of patient health heterogeneity, and a number of patients;
in accordance with a dynamic determination that the sufficient number of patients has been labelled, dynamically train the machine learning model based on the labelled patients by iteratively transmitting predictions by the machine learning model for marker values of a patient representation to be labelled;
receive selections of patients that are within the same dimensional space as a patient to be labelled; and
dynamically retrain a classifier of the machine learning model that labels a next batch of patients based on first labels of a first batch of patients and existing marker values for the patients of the electronic system.

9. The server of claim 8, wherein the server receives a randomly selected patient from the electronic system.

10. The server of claim 8, wherein the server is further configured with processor-executable instructions to vectorize the at least one of the healthcare record items associated with the selected focal patient and other patients in the system using all the statistical characteristics taken together (minimum, maximum, variance, skewness, range, mean, mode, median) of the sequence of a patient's data elements to extract new insights that are not apparent from the individual longitudinal raw data points.

11. The server of claim 8, wherein the server is further configured with processor-executable instructions to vectorize columns of the at least the healthcare record items associated with the selected focal patient and other patients in the system by at least one selected from a group including patient type, patient description, patient diagnosis, treatment type, treatment length, treatment description, and treatment category name list; and concatenating the vectorized columns together to form final vectors of the focal patient and other patients.

12. The server of claim 8, wherein the server is further configured with processor-executable instructions to assign a unique field index to a group of the labeled patients.

13. The server of claim 8, wherein the server is further configured with processor-executable instructions to assign one or more patients to an existing field.

14. The server of claim 7, wherein the server is further configured with processor-executable instructions to perform operations comprising:
receiving individual record data from a non-medical record source;
processing the individual record data in a large language model trained/fine-tuned to recognize and extract information that could be relevant to patient categorizing and treatment planning;
reformatting extracted personal data into format consistent with patient databases or compatible with the machine learning system; and
forming vectors from the extracted personal data.

15. A non-transitory processor-readable medium having stored thereon processor-executable instructions configured to cause a server to perform operations comprising:
receiving a selection of a focal patient from an electronic system stored in at least one storage device communicatively coupled to the server;
vectorizing at least one healthcare record item associated with the selected focal patient and other patients in the system, wherein the selected focal patient and the other patients in the system are without slot labels, and wherein the a dynamic number of slot labels are used to label one or more patients, with different patients having different slot labels, and wherein a machine learning model at the server is untrained in labeling the one or more patients with the slot labels;
determining at least one selected from the group including key healthcare record items and marker values from the healthcare record for each patient of the system;
forming vectors from at least one selected from the group including key healthcare record items and marker values;
concatenating the separate vectors together to form final vectors for the patients;
performing a similarity search using the final vectors to determine a group of similar patients from the vectorized patients of the system;
transmitting, via a communication network coupled to the server, the similar patients to a computer for display;
receiving a selection of the patients that are within a neighboring dimensional space as the focal patient;
labelling the selected patients in batch, wherein the selection of the patients and the focal patient within the same slots have similar slot labels;
determining whether a sufficient number of patients required to train the machine learning model has been labelled, wherein the sufficient number of patients is at least partially based on at least one selected from the group including a number of the patient record items, a measure of patient health heterogeneity, and a number of patients;
in accordance with a dynamic determination that the sufficient number of patients has been labelled, dynamically training the machine learning model based on the labelled patients by iteratively transmitting predictions by the machine learning model for marker values of a patient representation to be labelled;
receiving selections of patients that are within the same dimensional space as a patient to be labelled; and
dynamically retraining a classifier of the machine learning model that labels a next batch of patients based on first labels of a first batch of patients and existing marker values for the patients of the electronic system.

16. The non-transitory processor-readable medium of claim 13, wherein the stored processor-executable instructions are configured to cause a server to perform operations such that receiving a selection of a focal patient from an electronic system comprises receiving a randomly selected patient from the electronic system.

17. The non-transitory processor-readable medium of claim 13, wherein the stored processor-executable instructions are configured to cause a server to perform operations such that vectorizing at least one healthcare record item associated with the selected focal patient and other patients in the system comprises vectorizing the at least one of the healthcare record items associated with the selected focal patient and other patients in the system using all the statistical characteristics taken together (minimum, maximum, variance, skewness, range, mean, mode, median) of the sequence of a patient's data elements to extract new insights that are not apparent from the individual longitudinal raw data points.

18. The non-transitory processor-readable medium of claim 13, wherein the stored processor-executable instructions are configured to cause a server to perform operations such that vectorizing at least one healthcare record item associated with the selected focal patient and other patients in the system comprises vectorizing columns of the at least one of the healthcare record items associated with the selected focal patient and other patients in the system by at least one selected from a group including patient type, patient description, patient diagnosis, treatment type, treatment length, treatment description, and treatment category name list; and concatenating the vectorized columns together to form final vectors of the focal patient and other patients.

19. The non-transitory processor-readable medium of claim 13, wherein the stored processor-executable instructions are configured to cause a server to perform further operations comprising assigning a unique field index to a group of the labeled patients.

20. The non-transitory processor-readable medium of claim 13, wherein the stored processor-executable instructions are configured to cause a server to perform further operations comprising assigning one or more patients to an existing field.
